(19) United States
(12) Patent Application Publication
(10) Pub. No.: US 2005/0137145 A1
(43) Pub. Date: Jun. 23, 2005

(54) Metabolites of 1-dimethylamino-3-(3-methoxy-phenyl)-2-methyl-pentan-3-ol and their use in the treatment of urinary incontinence

(75) Inventors: Burkhard Kurth, Muenster (DE); Joerg Holenz, Barcelona (ES); Helmut Buschmann, Esplugues de Llobregat (ES); Oswald Zimmer, Wuerselen (DE); Thomas Christoph, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/999,219

(22) Filed: Nov. 30, 2004

Related U.S. Application Data
(63) Continuation of application No. PCT/EP03/05490, filed on May 26, 2003.

(30) Foreign Application Priority Data
May 30, 2002 (DE) 102 24 624.6

(51) Int. Cl. A61K 31/704

(52) U.S. Cl. 514/25

(57) ABSTRACT

1-phenyl-3-dimethylamino-propane compounds for producing a drug for treating urinary urgency or urinary incontinence and corresponding drugs and methods of treating or alleviating urinary urgency or urinary incontinence.

### CROSS REFERENCE TO RELATED APPLICATIONS

This application is a continuation of International Patent Application No. PCT/EP03/05490, filed May 26, 2003, designating the United States of America, and published in German as WO 03/101440 A1, the entire disclosure of which is incorporated herein by reference. Priority is claimed based on Federal Republic of Germany Patent Application No.102 24 624.6, filed May 30, 2002.

### FIELD OF THE INVENTION

The invention relates to metabolites of 1-dimethylamino-3-(3-methoxy-phenyl)-2-methyl-pentan-3-ol in the form of free bases and/or in the form of physiologically acceptable salts, to their use in the preparation of a medicament for the treatment of increased urinary urgency or urinary incontinence and also to corresponding medicaments, and to a method of treating increased urinary urgency or urinary incontinence.

### BACKGROUND OF THE INVENTION

Urinary incontinence is the involuntary leakage of urine. This occurs uncontrollably when the pressure inside the bladder exceeds the pressure required to close the ureter. Causes may include increased internal bladder pressure (e.g. as a result of detrusor instability), resulting in urge incontinence, as well as a reduced sphincter pressure (e.g. after childbirth or surgical interventions), resulting in stress incontinence. The detrusor is the roughly bundled multi-layer muscular system of the bladder wall, contraction of which leads to emptying of the bladder. The sphincter is the muscle that closes the urethra. Mixed forms of these types of incontinence occur, as well as so-called overflow incontinence (e.g. in benign prostatic hyperplasia) or reflex incontinence (e.g. after spinal cord damage). Further details will be found in Chutka, D. S. and Takahashi, P. Y., 1998, Drugs 560: 587-595.

Urinary urgency is the state of increased bladder muscle tension, with the aim of emptying the bladder (miction), as the capacity of the bladder is reached (or exceeded). This tension acts to stimulate miction. Increased urinary urgency is understood to mean especially the occurrence of premature or frequent, sometimes even painful urinary urgency and so-called urinary compulsion. This results in markedly more frequent miction. Causes may be, inter alia, inflammations of the bladder and neurogenic bladder disorders as well as bladder tuberculosis. Not all causes have yet been clarified, however.

Increased urinary urgency and urinary incontinence are extremely unpleasant and there is a clear need in persons affected by these indications to achieve an improvement that is as long-term as possible.

Increased urinary urgency and, especially, urinary incontinence are usually treated with medicaments, using substances that are involved in the reflexes of the lower urinary tract (Wein, A. J., 1998, Urology 51 (Suppl. 21): 43-47). In most cases these are medicaments that have an inhibiting action on the detrusor muscle, which is responsible for the internal bladder pressure. Such medicaments are, for example, parasympatholytics such as oxybutynin, propiverine or tolterodine, tricyclic antidepressants such as imipramine or muscle relaxants such as flavoxate. Other medicaments, which in particular increase the resistance of the urethra or of the bladder neck, show affinities for the α-adrenoreceptors, such as ephedrine, for β-adrenoreceptors, such as clenbutarol, or are hormones, such as oestradiol. Some opioids, diarylmethyl-piperazines and -piperidines are also described for this indication in WO 93/15062.

With the indications being considered here it is to be noted that medicaments are generally administered over the very long term and that those affected, in contrast to many situations in which analgesics are used, are faced with a very unpleasant but not intolerable situation. Care should therefore be taken—even more so than in the case of analgesics—to avoid side-effects if the affected person does not wish to replace one trouble with another. Also, analgesic effects are largely undesirable in the case of permanent treatment for urinary incontinence.

### SUMMARY OF THE INVENTION

One object of the present invention was, therefore, to find substances which are useful in the treatment of increased urinary urgency or urinary incontinence and which at the same time, at the effective doses, preferably exhibit fewer side-effects and/or analgesic effects than are known from the prior art.

Surprisingly, it has now been found that 1-dimethylamino-3-(3-methoxy-phenyl)-2-methyl-pentan-3-ol and its metabolites possess an outstanding action on bladder function and consequently are highly suitable for the treatment of corresponding disorders.

Accordingly, the invention relates to the use of (2RS,3RS)-1-dimethylamino-3-(3-methoxy-phenyl)-2-methyl-pentan-3-ol, (+)-(2R,3R)-1-dimethylamino-3-(3-methoxy-phenyl)-2-methyl-pentan-3-ol, (−)-(2S,3S)-1-dimethylamino-3-(3-methoxy-phenyl)-2-methyl-pentan-3-ol, (2RS,3RS)-3-(3-dimethylamino-1-ethyl-1-hydroxy-2-methyl-propyl)-phenol, (+)-(2R,3R)-3-(3-dimethylamino-1-ethyl-1-hydroxy-2-methyl-propyl)-phenol, (−)-(2S,3S)-3-(3-dimethylamino-1-ethyl-1-hydroxy-2-methyl-propyl)-phenol, 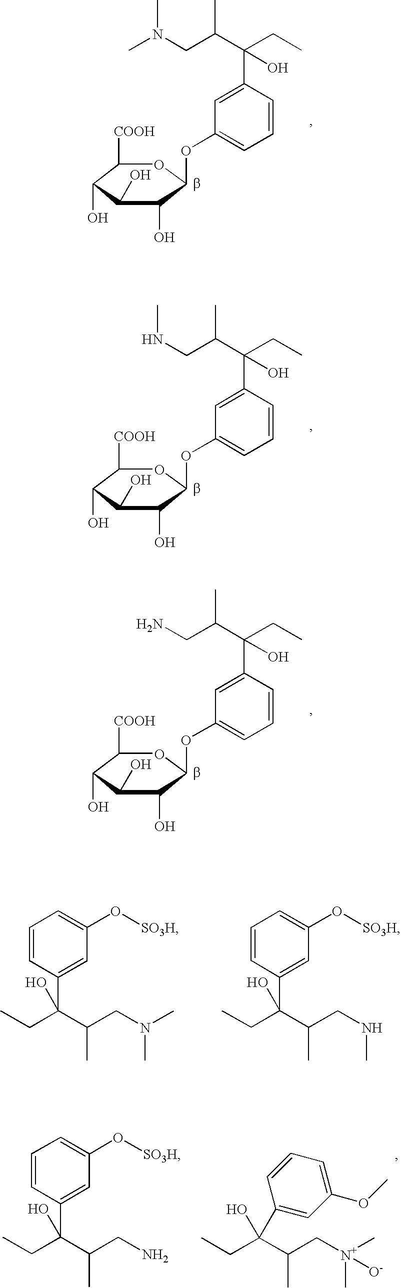 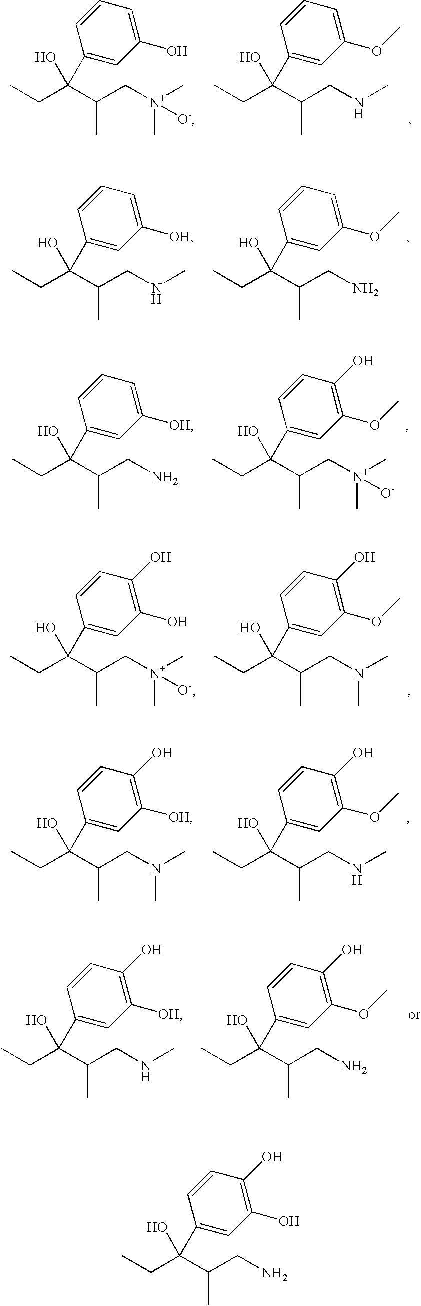
optionally in the form of their racemates, their pure stereoisomers, especially enantiomers or diastereoisomers, or in the form of mixtures of the stereoisomers, especially of the enantiomers or diastereoisomers, in any desired mixing ratio;

in the prepared form or in the form of their acids or their bases or in the form of their salts, especially the physiologically acceptable salts, or in the form of their solvates, especially the hydrates;

in the preparation of a medicament for the treatment of increased urinary urgency or urinary incontinence as well as for the treatment of these conditions.

It is particularly preferred for the compounds that are used to be in the form of R,R, preferably 2R,3R, stereoisomers.

Surprisingly, it has been found that the mentioned substances have a markedly positive effect on particular physiological parameters that are of importance in the case of increased urinary urgency or urinary incontinence, either on the threshold pressure, on the inter-contraction interval, or the reduction in rhythmic bladder contractions, and/or on the bladder capacity. Each of these changes can mean a marked easing in the symptomatic profile of affected patients.

The mentioned compounds can also be used in the preparation of medicaments for the treatment of pain.

(2RS,3RS)-1-Dimethylamino-3-(3-methoxy-phenyl)-2-methyl-pentan-3-ol, (+)-(2R,3R)-1-dimethylamino-3-(3-methoxy-phenyl)-2-methyl-pentan-3-ol, (−)-(2S,3S)-1-dimethylamino-3-(3-methoxy-phenyl)-2-methyl-pentan-3-ol, (2RS,3RS)-3-(3-dimethylamino-1-ethyl-1-hydroxy-2-methyl-propyl)-phenol, (+)-(2R,3R)-3-(3-dimethylamino-1-ethyl-1-hydroxy-2-methyl-propyl)-phenol, (−)-(2S,3S)-3-(3-dimethylamino-1-ethyl-1-hydroxy-2-methyl-propyl)-phenol and their preparation are known from DE 44 26 245 A1, or U.S. Pat. No. 6,248,737.

In the context of this invention, alkyl and cycloalkyl radicals are understood as meaning saturated and unsaturated (but not aromatic), branched, unbranched and cyclic hydrocarbons, which can be unsubstituted or mono- or poly-substituted. C₁₋₂-Alkyl means C1- or C2-alkyl, C₁₋₃-alkyl means C1-, C2- or C3-alkyl, C₁₋₄-alkyl means C1-, C2-, C3- or C4-alkyl, C₁₋₅-alkyl means C1-, C2-, C3-, C4- or C5-alkyl, C₁₋₆-alkyl means C1-, C2-, C3-, C4-, C5- or C6-alkyl, C₁₋₇-alkyl means C1-, C2-, C3-, C4-, C5-, C6- or C7-alkyl, C₁₋₈-alkyl means C1-, C2-, C3-, C4-, C5-, C6-, C7- or C8-alkyl, C₁₋₁₀-alkyl means C1-, C2-, C3-, C4-, C5-, C6-, C7-, C8-, C9- or C10-alkyl and C₁₋₁₈-alkyl means C1-, C2-, C3-, C4-, C5-, C6-, C7-, C8-, C9-, C10-, C11-, C12-, C13-, C14-, C15-, C16-, C17 or C18-alkyl. Furthermore, C₃₋₄-cycloalkyl means C3- or C4-cycloalkyl, C₃₋₅-cycloalkyl means C3-, C4- or C5-cycloalkyl, C₃₋₆-cycloalkyl means C3-, C4-, C5- or C6-cycloalkyl, C₃₋₇-cycloalkyl means C3-, C4-, C5-, C6- or C7-cycloalkyl, C₃₋₈-cycloalkyl means C3-, C4-, C5-, C6-, C7 or C8-cycloalkyl, C₄₋₅-cycloalkyl means C4- or C5-cycloalkyl, C₄₋₆-cycloalkyl means C4-, C5- or C6-cycloalkyl, C₄₋₇-cycloalkyl means C4-, C5-, C6- or C7-cycloalkyl, C₅₋₆-cycloalkyl means C5- or C6-cycloalkyl and C₅₋₇-cycloalkyl means C5-, C6- or C7-cycloalkyl. With respect to cycloalkyl, the term also includes saturated cycloalkyls in which one or 2 carbon atoms are replaced by a heteroatom, S, N or O. However, the term cycloalkyl also includes especially mono- or poly-unsaturated, preferably monounsaturated, cycloalkyls without a heteroatom in the ring, provided the cycloalkyl is not an aromatic system. The alkyl and cycloalkyl radicals are preferably methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl, cyclooctyl, and also adamantyl, CHF₂, CF₃ or CH₂OH, as well as pyrazolinone, oxopyrazolinone, [1,4]dioxane or dioxolane.

In connection with alkyl and cycloalkyl—unless expressly defined otherwise—the term substituted here is understood in the context of this invention as meaning the substitution of at least one hydrogen radical (optionally a plurality of hydrogen radicals) by F, Cl, Br, I, NH₂, SH or OH, where “polysubstituted” or “substituted” in the case of multiple substitution is to be understood as meaning that the substitution occurs several times with the same or different substituents both on different and on the same atoms, for example three times on the same C atom, as in the case of CF₃, or at different places, as in the case of —CH(OH)—CH═CH—CHCl₂. Particularly preferred substituents here are F, Cl and OH. In respect of cycloalkyl, the hydrogen radical can also be replaced by OC₁₋₃-alkyl or C₁₋₃-alkyl (in each case mono- or poly-substituted or unsubstituted), especially methyl, ethyl, n-propyl, isopropyl, CF₃, methoxy or ethoxy.

The term (CH₂)₃₋₆ is to be understood as meaning —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—, (CH₂)₁₋₄ is to be understood as meaning —CH₂—, —CH₂—CH₂—, —CH₂—CH₂—CH₂— and —CH₂—CH₂—CH₂—CH₂—, (CH₂)₄₋₅ is to be understood as meaning —CH₂—CH₂—CH₂—CH₂— and —CH₂—CH₂—CH₂—CH₂—CH₂—, etc.

An aryl radical is understood as meaning ring systems having at least one aromatic ring but without heteroatoms in even only one of the rings. Examples are phenyl, naphthyl, fluoranthenyl, fluorenyl, tetralinyl or indanyl, especially 9H-fluorenyl or anthracenyl radicals, which can be unsubstituted or mono- or poly-substituted.

A heteroaryl radical is understood as meaning heterocyclic ring systems having at least one unsaturated ring, which contain one or more heteroatoms from the group nitrogen, oxygen and/or sulfur and can also be mono- or poly-substituted. Examples which may be mentioned from the group of heteroaryls are furan, benzofuran, thiophene, benzothiophene, pyrrole, pyridine, pyrimidine, pyrazine, quinoline, isoquinoline, phthalazine, benzo[1,2,5]thiadiazole, benzothiazole, indole, benzotriazole, benzodioxolane, benzodioxane, carbazole, indole and quinazoline.

In connection with aryl and heteroaryl, substituted here is understood as meaning substitution of the aryl or heteroaryl with R²², OR²² a halogen, preferably F and/or Cl, a CF₃, a CN, an NO₂, an NR²³R²⁴, a C₁₋₆-alkyl (saturated), a C₁₋₆-alkoxy, a C₃₋₈-cycloalkoxy, a C₃₋₈-cycloalkyl or a C₂₋₆-alkylene.

The radical R²² here represents H, a C₁₋₁₀-alkyl radical, preferably a C₁₋₆-alkyl radical, an aryl or heteroaryl radical or an aryl or heteroaryl radical bonded via C₁₋₃-alkyl, saturated or unsaturated, or via a C₁₋₃-alkylene group, wherein these aryl and heteroaryl radicals may not themselves be substituted by aryl or heteroaryl radicals,

the radicals R²³ and R²⁴, which are identical or different, represent H, a C₁₋₁₀-alkyl radical, preferably a C₁₋₆-alkyl radical, an aryl radical, a heteroaryl radical or an aryl or heteroaryl radical bonded via C₁₋₃-alkyl, saturated or unsaturated, or via a C₁₋₃-alkylene group, wherein these aryl and heteroaryl radicals may not themselves be substituted by aryl or heteroaryl radicals,

or the radicals R²³ and R²⁴ together represent CH₂CH₂OCH₂CH₂, CH₂CH₂NR²⁵CH₂CH₂ or (CH₂)₃₋₆, and

the radical R²⁵ represents H, a C₁₋₁₀-alkyl radical, preferably a C₁₋₆-alkyl radical, an aryl or heteroaryl radical or an aryl or heteroaryl radical bonded via C₁₋₃-alkyl, saturated or unsaturated, or via a C₁₋₃-alkylene group, wherein these aryl and heteroaryl radicals may not themselves be substituted by aryl or heteroaryl radicals.

The term salt is to be understood as meaning any form of the active ingredient according to the invention in which the active ingredient assumes an ionic form or is charged and is coupled with a counter-ion (a cation or anion) or is in solution. The term is also to be understood as meaning complexes of the active ingredient with other molecules and ions, especially complexes complexed via ionic interactions. In particular, the term is understood as meaning (and this is also a preferred embodiment of this invention) physiologically acceptable salts, especially physiologically acceptable salts with cations or bases and physiologically acceptable salts with anions or acids or a salt formed with a physiologically acceptable acid or a physiologically acceptable cation.

The term of the physiologically acceptable salt with anions or acids is understood in the context of this invention as meaning salts of at least one of the compounds according to the invention—in most cases protonated, for example at the nitrogen—as the cation with at least one anion, which salts are physiologically acceptable—especially when used in humans and/or mammals. In particular, the term is understood in the context of this invention as meaning the salt formed with a physiologically acceptable acid, namely salts of the particular active ingredient with inorganic or organic acids which are physiologically acceptable—especially when used in humans and/or mammals. Examples of physiologically acceptable salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydro1λ⁶-benzo[d]isothiazol-3-one (saccharic acid), monomethylsebacic acid, 5-oxo-proline, hexane-1-sulfonic acid, nicotinic acid, 2-, 3- or 4-aminobenzoic acid, 2,4,6-trimethyl-benzoic acid, a-liponic acid, acetylglycine, acetylsalicylic acid, hippuric acid and/or aspartic acid. The hydrochloride salt is particularly preferred.

The term of the salt formed with a physiologically acceptable acid is understood in the context of this invention as meaning salts of the particular active ingredient with inorganic or organic acids which are physiologically acceptable—especially when used in humans and/or mammals. The hydrochloride is particularly preferred. Examples of physiologically acceptable acids are: hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid, 1,1-dioxo-1,2-dihydro1λ⁶-benzo[d]isothiazol-3-one (saccharic acid), monomethylsebacic acid, 5-oxo-proline, hexane-1-sulfonic acid, nicotinic acid, 2-, 3- or 4-aminobenzoic acid, 2,4,6-trimethyl-benzoic acid, a-liponic acid, acetylglycine, acetylsalicylic acid, hippuric acid and/or aspartic acid.

The term of the physiologically acceptable salt with cations or bases is understood in the context of this invention as meaning salts of at least one of the compounds according to the invention—in most cases of a (deprotonated) acid—as the anion with at least one cation, preferably an inorganic cation, which salts are physiologically acceptable—especially when used in humans and/or mammals. The salts of the alkali metals and alkaline earth metals and also NH₄⁺ are particularly preferred, but especially (mono-) or (di-)sodium, (mono-) or (di-)potassium, magnesium or calcium salts.

The term of the salt formed with a physiologically acceptable cation is understood in the context of this invention as meaning salts of at least one of the particular compounds as the anion with at least one inorganic cation which is physiologically acceptable—especially when used in humans and/or mammals. The salts of the alkali metals and alkaline earth metals and also NH₄⁺ are particularly preferred, but especially (mono-) or (di-)sodium, (mono-) or (di-)potassium, magnesium or calcium salts.

The invention further provides metabolites of 1-dimethylamino-3-(3-methoxy-phenyl)-2-methyl-pentan-3-ol selected from 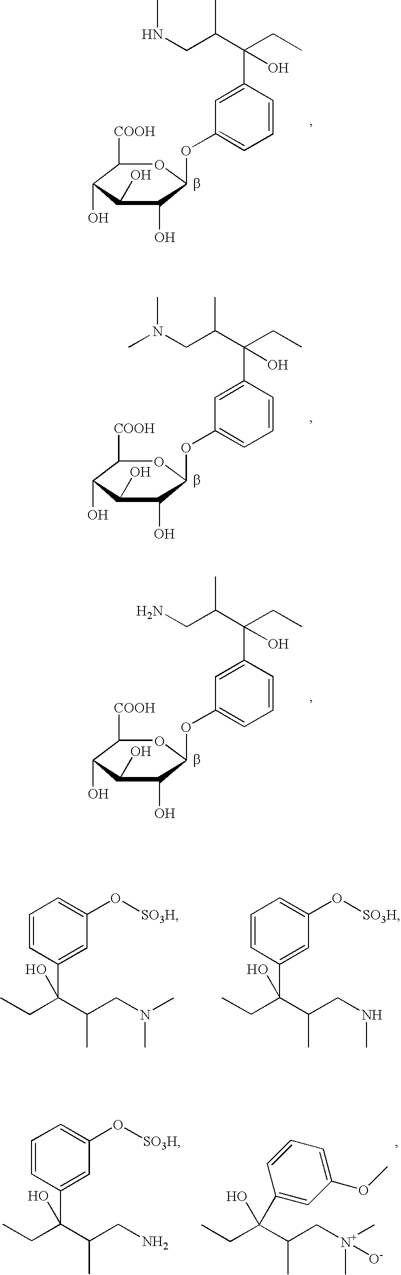 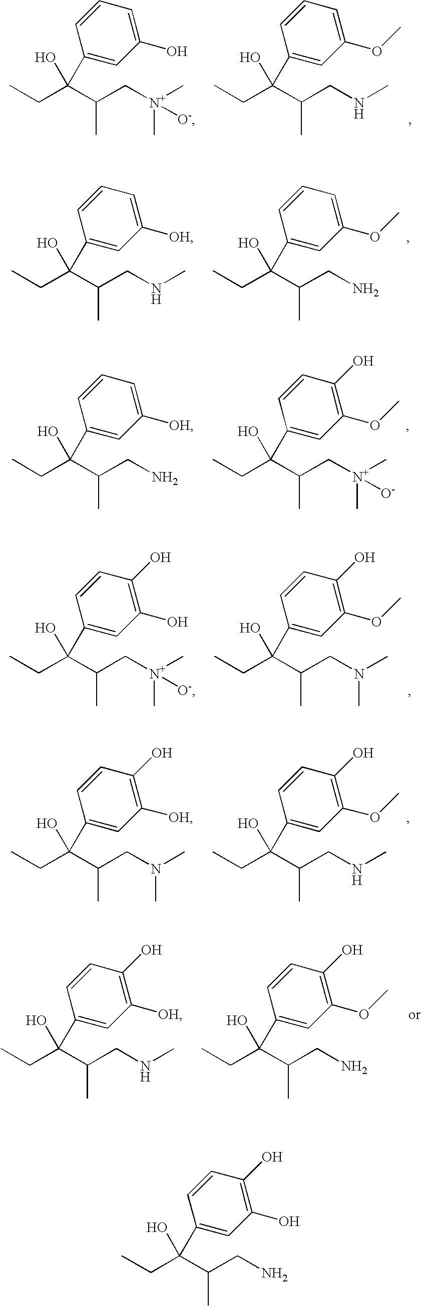
optionally in the form of their racemates, their pure stereoisomers, especially enantiomers or diastereoisomers, or in the form of mixtures of the stereoisomers, especially of the enantiomers or diastereoisomers, in any desired mixing ratio;

in the prepared form or in the form of their acids or their bases or in the form of their salts, especially the physiologically acceptable salts, or in the form of their solvates, especially the hydrates.

Particular preference is given to metabolites according to the invention that are in the form of R,R, preferably 2R,3R, stereoisomers.

The metabolites according to the invention are physiologically harmless. The invention accordingly further provides a medicament, preferably for the treatment of increased urinary urgency or urinary incontinence, comprising as active ingredient at least one compound selected from (2RS,3RS)-1-dimethylamino-3-(3-methoxy-phenyl)-2-methyl-pentan-3-ol, (+)-(2R,3R)-1-dimethylamino-3-(3-methoxy-phenyl)-2-methyl-pentan-3-ol, (−)-(2S,3S)-1-dimethylamino-3-(3-methoxy-phenyl)-2-methyl-pentan-3-ol, (2RS,3RS)-3-(3-dimethylamino-1-ethyl-1-hydroxy-2-methyl-propyl)-phenol, (+)-(2R,3R)-3-(3-dimethylamino-1-ethyl-1-hydroxy-2-methyl-propyl)-phenol, (−)-(2S,3S)-3-(3-dimethylamino-1-ethyl-1-hydroxy-2-methyl-propyl)-phenol, 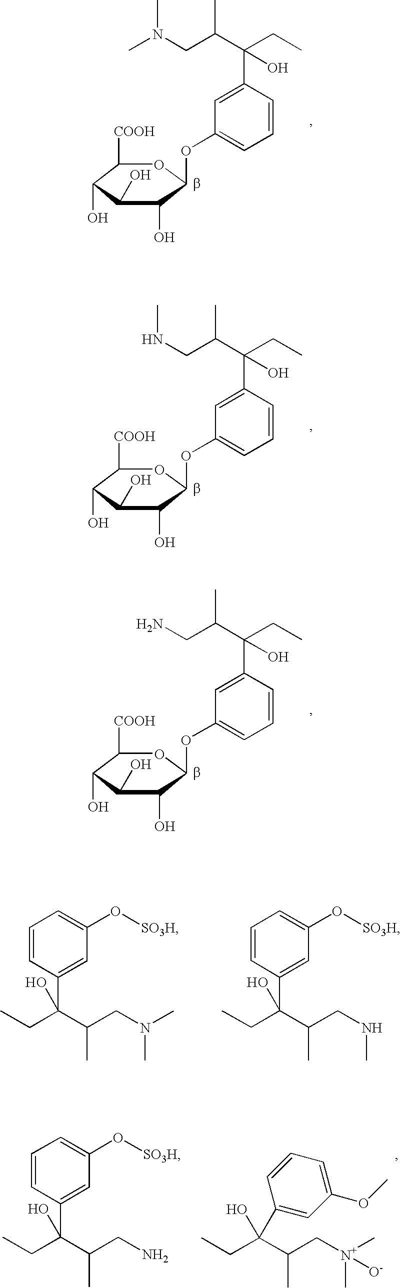 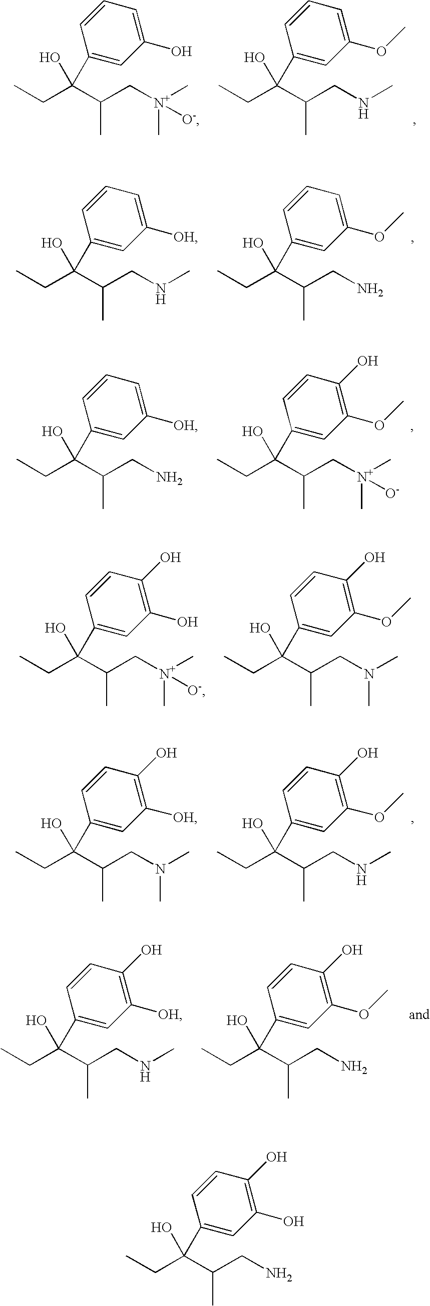
optionally in the form of their racemates, their pure stereoisomers, especially enantiomers or diastereoisomers, or in the form of mixtures of the stereoisomers, especially of the enantiomers or diastereoisomers, in any desired mixing ratio;
in the prepared form or in the form of their acids or their bases or in the form of their salts, especially the physiologically acceptable salts, or in the form of their solvates, especially the hydrates;
and optionally additives and/or auxiliary substances.

It is particularly preferred for the compounds that are present to be in the form of R,R, preferably 2R,3R, stereoisomers.

Suitable additives and/or auxiliary substances in the context of this invention are any substances from the prior art that are known to the person skilled in the art for obtaining galenical formulations. The choice of auxiliary substances and the amounts thereof to be used depend on whether the medicament is to be administered orally, intravenously, intraperitoneally, intradermally, intramuscularly, intranasally, buccally or locally. Preparations in the form of tablets, chewing tablets, dragées, capsules, granules, drops, juices or syrups are suitable for oral administration, and solutions, suspensions, readily reconstitutable dry preparations and sprays are suitable for parenteral and topical administration and administration by inhalation. A further possibility is suppositories for administration in the rectum. Examples of suitable percutaneous forms of administration are administration in a depot in dissolved form, in a carrier film or in a plaster, optionally with the addition of agents promoting penetration through the skin. Examples of auxiliary substances and additives for oral forms of administration are disintegrators, lubricants, binders, fillers, mould release agents, optionally solvents, flavourings, sugars, especially carriers, diluents, colourings, antioxidants, etc. For suppositories there may be used, inter alia, waxes or fatty acid esters, and for parenteral administration agents there may be used carriers, preservatives, suspension aids, etc. The amounts of active ingredient to be administered to patients vary in dependence on the weight of the patient, the mode of administration and the severity of the disorder. The compounds according to the invention can be released in a delayed manner from forms of preparation for oral, rectal or percutaneous administration. In the case of the indication according to the invention, corresponding retard formulations, especially in the form of a “once-daily” preparation that must be taken only once per day, are particularly preferred.

Also preferred are medicaments that comprise at least from 0.05 to 90.0% of the active ingredient, especially doses having low activity, in order to avoid side-effects or analgesic effects. Usually, from 0.1 to 5000 mg/kg, especially from 1 to 500 mg/kg, preferably from 2 to 250 mg/kg body weight of at least one of the compounds according to the invention or used according to the invention are administered. However, it is likewise preferred and usual to administer from 0.01 to 5 mg/kg, preferably from 0.03 to 2 mg/kg, especially from 0.05 to 1 mg/kg body weight.

Auxiliary substances may be, for example: water, ethanol, 2-propanol, glycerol, ethylene glycol, propylene glycol, polyethylene glycol, polypropylene glycol, glucose, fructose, lactose, saccharose, dextrose, molasses, starch, modified starch, gelatin, sorbitol, inositol, mannitol, microcrystalline cellulose, methyl cellulose, carboxymethyl cellulose, cellulose acetate, shellac, cetyl alcohol, polyvinylpyrrolidone, paraffins, waxes, natural and synthetic gums, gum arabic, alginates, dextran, saturated and unsaturated fatty acids, stearic acid, magnesium stearate, zinc stearate, glyceryl stearate, sodium lauryl sulfate, edible oils, sesame oil, coconut oil, groundnut oil, soybean oil, lecithin, sodium lactate, polyoxyethylene and polyoxypropylene fatty acid esters, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talcum, kaolin, pectin, crospovidone, agar and bentonite.

The preparation of the medicaments and pharmaceutical compositions according to the invention is carried out by means of agents, devices, methods and processes which are well known in the art of pharmaceutical formulation, as are described, for example, in “Remington's Pharmaceutical Sciences”, ed. A. R. Gennaro, 17th Ed., Mack Publishing Company, Easton, Pa. (1985), especially in Part 8, Chapters 76 to 93.

Thus, for example, for a solid formulation, such as a tablet, the active ingredient of the medicament, i.e. a compound according to the invention or used according to the invention, or a pharmaceutically acceptable salt thereof, can be granulated with a pharmaceutical carrier, for example conventional tablet constituents, such as maize starch, lactose, saccharose, sorbitol, talcum, magnesium stearate, dicalcium phosphate or pharmaceutically acceptable gums, and pharmaceutical diluents, for example water, in order to form a solid composition which comprises a compound according to the invention, or a pharmaceutically acceptable salt thereof, in homogeneous distribution. Homogeneous distribution is here understood as meaning that the active ingredient is distributed uniformly throughout the composition, so that the latter can readily be divided into unit dose forms, such as tablets, pills or capsules, that have equal activity. The solid composition is then divided into unit dose forms. The tablets or pills of the medicament according to the invention or of the compositions according to the invention may also be coated or otherwise compounded in order to provide a delayed-release dosage form. Suitable coating agents are, inter alia, polymeric acids and mixtures of polymeric acids with materials such as, for example, shellac, cetyl alcohol and/or cellulose acetate.

Although the medicaments according to the invention exhibit only few side-effects, it may be advantageous, for example in order to avoid particular forms of dependency, to use morphine antagonists, especially naloxone, naltrexone and/or levallorphan, in addition to the compounds according to the general formula I.

The invention furthermore relates also to a method of treating increased urinary urgency or urinary incontinence, in which the compounds used according to the invention are employed.

The compounds whose preparation is not known from DE 44 26245 A1 are synthesised as follows:

Process A: For the preparation of 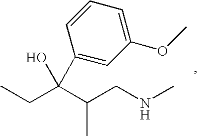
in a modification of the synthesis in Example 1 of DE 44 26 245, or U.S. Pat. No. 6,248,737, first of all 1-methyl-1-benzylamino-2-methyl-pentan-3-one is used instead of 1-dimethylamino-2-methyl-pentan-3-one. 1-Methyl-1-benzylamino-2-methyl-pentan-3-one can be bought or prepared from 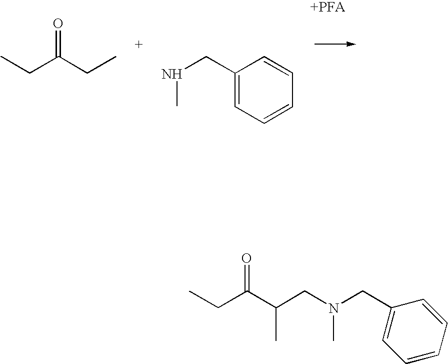
(PFA=paraformaldehyde). Then the further procedure is analogous to Examples 1 and 2. The resulting compound is then treated with H₂, Pd/C, EtOH and base, so that the benzyl group on the nitrogen is removed.

Process B: For the preparation of 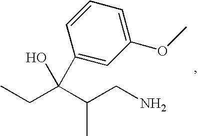
in a modification of the synthesis in Example 1 of DE 44 26 245, or U.S. Pat. No. 6,248,737, first of all 1-dibenzylamino-2-methyl-pentan-3-one is used instead of 1-dimethylamino-2-methyl-pentan-3-one. 1-Dibenzylamino-2-methyl-pentan-3-one can be purchased or prepared from 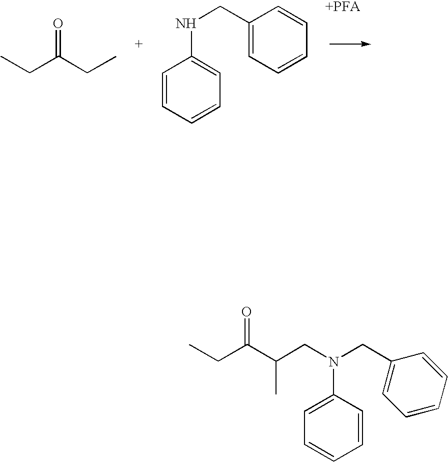
(PFA=paraformaldehyde). Then the further procedure is analogous to Examples 1 and 2. The resulting compound is then treated in two steps, in each case once, with H₂, Pd/C, EtOH and base, so that the benzyl groups are removed successively from the nitrogen.

Process C: For the preparation of 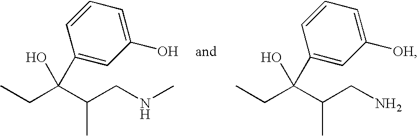
first of all 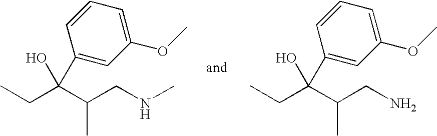
are prepared according to processes A and B. The compounds are then boiled for 2 days at 120° C. in the presence of in most cases 8 equivalents of DIBAH (=diisobutylaluminium hydride) in toluene. The desired hydroxy compound is formed.

Process D: First of all, 3-(3-dimethylamino-1-ethyl-1-hydroxy-2-methyl-propyl)-phenol is prepared according to DE 44 26 245, or U.S. Pat. No. 6,248,737, or 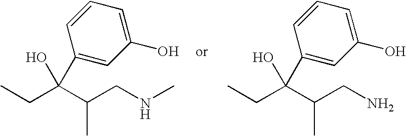
is prepared according to process C. Those compounds are then treated with dicyclohexylcarbodiimide (DCC) in H₂SO₄ and DMF. There are formed:

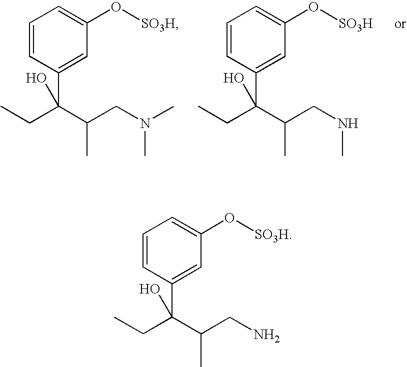

Process E: First of all, 3-(3-dimethylamino-1-ethyl-1-hydroxy-2-methyl-propyl)-phenol is prepared according to DE 44 26 245, or U.S. Pat. No. 6,248,737, or 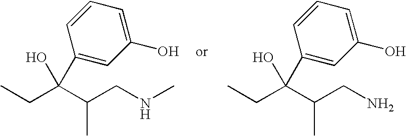
is prepared according to process C. Those compounds are then treated with 3,4,5-tri-O-acetyl-1-a-bromo-D-glucoronic acid methyl ester 1. with LiOH and 2. with HOAc. Purification is carried out by means of HPLC separation. There are formed:

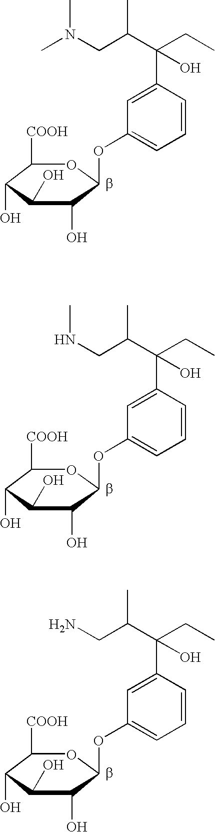

In general, purification and enantiomeric separation are carried out in all the mentioned processes at different stages by means of CC and, predominantly, HPLC.

Process F: For the preparation of 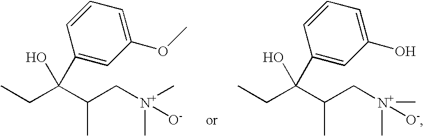
first of all 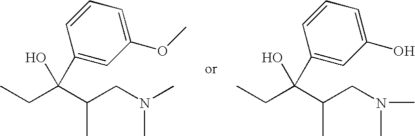
are prepared according to DE 44 26 245, or U.S. Pat. No. 6,248,737. Those compounds are then oxidized with peroxide in alcoholic solution, preferably in MeOH.

Process G for all metabolites having a hydroxy group in the para-position on the phenol ring. It is possible to prepare these compounds in a simple manner according or analogously to the procedures of DE 44 26 245, or U.S. Pat. No. 6,248,737, or according to the processes described hereinbefore, especially using known protecting groups for the hydroxy group.

Process H (alternative) for all metabolites: 1-Dimethylamino-3-(3-methoxy-phenyl)-2-methyl-pentan-3-ol is dissolved in TRIS/HCl buffer, pH 7.4. MgCl and, optionally, the other necessary co-factors for CytP450 known in the literature are added, and incubation is carried out at 37° C. with a mixture of sub-types of Cytochrom P450 (CytP450), especially CytP450 3A4 (N-demethylation) and/or CytP450 2D6 (O-demethylation and hydroxylation). The batch is then separated by HPLC and the metabolites in the fractions are identified by NMR and hence isolated.

The following Examples are intended to illustrate the invention, without the subject-matter of the invention being limited thereto.

### EXAMPLES

### Example 1: List of Tested Substances

The compounds tested in respect of their effectiveness are listed below:

| | Comp. |
|---|---|
| Name | No. |
| (2RS,3RS)-1-Dimethylamino-3-(3-methoxy-phenyl)-2-methyl- | 1 |
| pentan-3-ol, hydrochloride | |
| (+)-(2R,3R)-1-Dimethylamino-3-(3-methoxy-phenyl)-2-methyl- | 2 |
| pentan-3-ol, hydrochloride | |
| (−)-(2S,3S)-1-Dimethylamino-3-(3-methoxy-phenyl)-2-methyl- | 21 |
| pentan-3-ol, hydrochloride | |
| (2RS,3RS)-3-(3-Dimethylamino-1-ethyl-1-hydroxy-2-methyl- | 7 |
| propyl)-phenol, hydrochloride | |

### Example 2: Cystometry Test System on the Conscious Naive Rat

Cystometric investigations were carried out on naive female Sprague-Dawley rats according to the method of Ishizuka et al. ((1997), Naunyn-Schmiedeberg's Arch. Pharmacol. 355: 787-793). Three days after the implantation of bladder and venous catheters, the animals, which were free to move, were studied in the conscious state. The bladder catheter was attached to a pressure transducer and an injection pump. The animals were placed in metabolic cages which allowed the volume of urine to be measured. Physiological saline solution was infused into the emptied bladder (10 ml/h) and the bladder pressure and miction volume were recorded continuously. After a stabilization phase, a 20-minute phase characterised by normal, reproducible miction cycles was recorded. The following parameters, inter alia, were determined:
threshold pressure (TP, bladder pressure immediately before miction),
bladder capacity (BC, residual volume after previous miction plus volume of solution infused during the filling phase),
inter-contraction interval (ICI, the time interval between mictions).

An increase in the threshold pressure (TP) indicates an important therapeutic activity in one of the indications according to the invention. The inter-contraction interval (ICI) is also an important parameter for measuring the physiological effectiveness of a substance in the treatment of urinary incontinence, as is the bladder capacity (BC). Because the causes of the symptoms of these disorders are highly heterogeneous, it is not necessary for all three parameters to be influenced positively in order for effectiveness to exist. It is, therefore, entirely satisfactory for a positive effect to be determined in only one of these parameters in order to be usable in urinary incontinence or increased urinary urgency.

After three reproducible miction cycles had been recorded as the preliminary value, the test substances 1 (1.0 mg/kg), 2 (0.1; 0.3 and 0.5 mg/kg), 21 (0.5 mg/kg) and 7 (0.3 mg/kg) were administered i.v. in the vehicle=0.9% NaCl and the effect on the cystometric parameters was recorded for 90 to 120 minutes. At the maximum activity, the mean of 3 miction cycles was determined and indicated as the percentage change relative to the preliminary value (Table 1).

TABLE 1
Effect of the test substances on the cystometric parameters
(change relative to the preliminary value [%]);

| | TP | BC | ICI |
|---|---|---|---|
| Compound: | threshold | bladder | inter-contraction |
| (concentration) | pressure | capacity | interval |
| 1 | | | |
| 1.0 mg/kg i.v. | +94%** | +31%*** | +42% |
| (n = 9) | | | |
| 2 | | | |
| 0.1 mg/kg i.v. | +28.5%** | +7.8% | +15.6% |
| (n = 5) | | | |
| 0.3 mg/kg i.v. | +122%** | +33%* | +28%* |
| (n = 8) | | | |
| 0.5 mg/kg i.v. | +77.5%** | +20.6%* | +28.6%** |
| (n = 9) | | | |
| 21 | | | |
| 0.5 mg/kg i.v. | −1.1% | +3% | +10% |
| (n = 8) | | | |
| 7 | | | |
| 0.3 mg/kg i.v. | +95%** | +32%* | +28%* |
| (n = 7) | | | |

n is the number of test animals;
significance (Student T test):
*p < 0.05;
**p < 0.01;
***p < 0.001.

The tested substances show a positive effect on bladder regulation and are therefore suitable for the treatment of urinary incontinence.

It has been found, inter alia, that of the enantiomers of the racemic compound 1, the (+)-enantiomer (compound 2) is very effective (and accordingly is a particularly preferred compound of this invention), while the (−)-enantiomer (compound 21) does not make such a strong contribution to the action.

### Example 3: Parenteral Form of Administration

1 g of compound 2 is dissolved at room temperature in 1 litre of water for injection purposes and then adjusted to isotonic conditions by addition of NaCl.

1. A method for the treatment of increased urinary urgency or urinary incontinence in a mammal, said method comprising the step of administering an effective amount of a compound selected from the group consisting of: 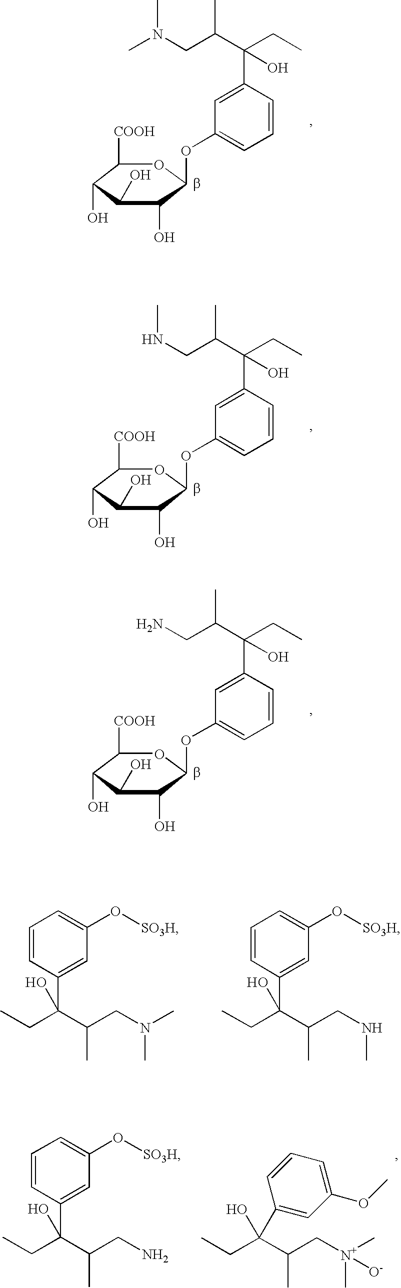 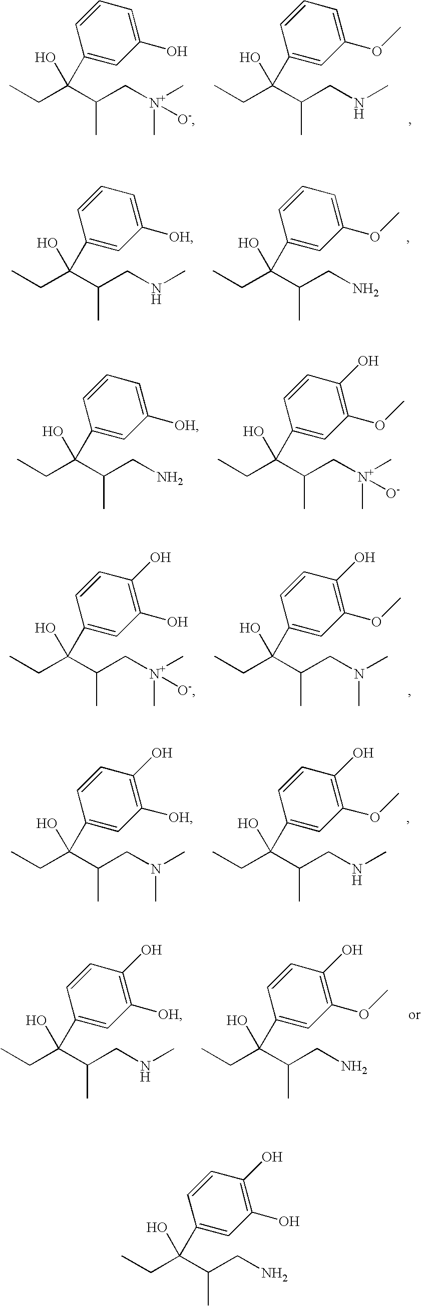
or a salt thereof with a physiologically tolerated acid.

2. The method of claim 1, wherein said compound is present in the form of a pure enantiomer or diastereoisomer.

3. The method of claim 1, wherein said compound is present in the form of a mixture of stereoisomers.

4. The method of claim 1, wherein said compound is present in the form of a racemic mixture.

5. The method of claim 1, wherein said compound is present in the form of a free base.

6. The method of claim 1, wherein said compound is present in the form of a solvate.

7. The method of claim 1, wherein said compound is present in the form of a hydrate.

8. The method of claim 1, wherein said compound is present in the form of an R,R stereoisomer.

9. The method of claim 1, wherein said compound is present in the form of a 2R,3R stereoisomer.

10. A method for alleviating increased urinary urgency in a mammal, said method comprising the step of administering an effective amount of a compound selected from the group consisting of: 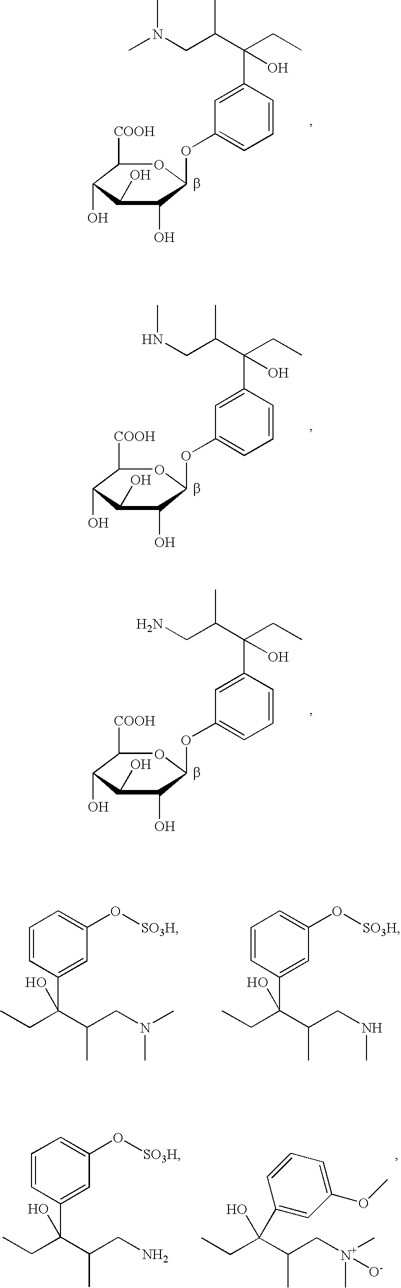 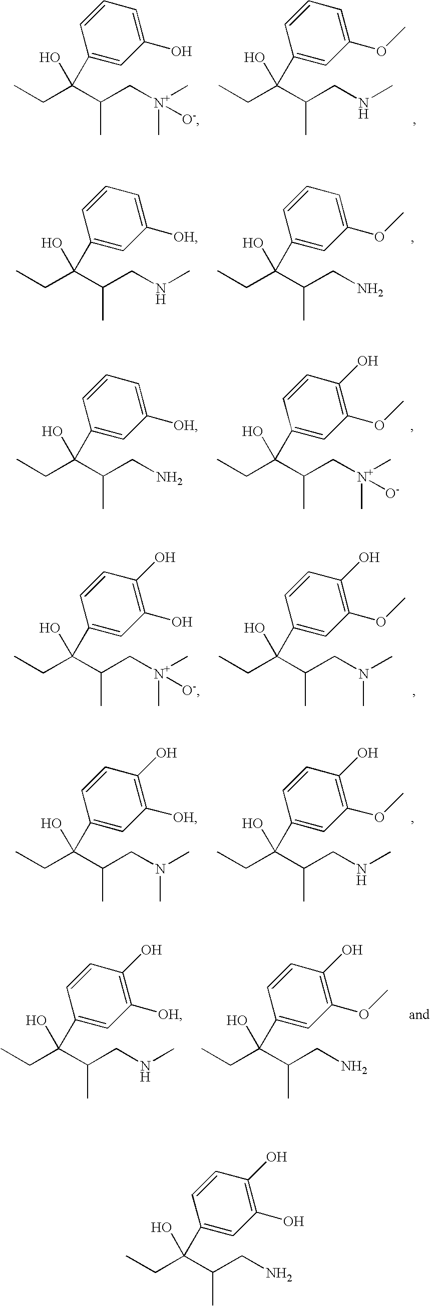
or a salt thereof with a physiologically tolerated acid.

11. The method of claim 10, wherein said compound is present in the form of a pure enantiomer or diastereoisomer.

12. The method of claim 10, wherein said compound is present in the form of a mixture of stereoisomers.

13. The method of claim 10, wherein said compound is present in the form of a racemic mixture.

14. The method of claim 10, wherein said compound is present in the form of a free base.

15. The method of claim 10, wherein said compound is present in the form of a solvate.

16. The method of claim 10, wherein said compound is present in the form of a hydrate.

17. The method of claim 10, wherein said compound is present in the form of an R,R stereoisomer.

18. The method of claim 10, wherein said compound is present in the form of a 2R,3R stereoisomer.
